(19) United States
(12) Patent Application Publication
(10) Pub. No.: US 2008/0177116 A1
(43) Pub. Date: Jul. 24, 2008

(54) 1-Bromopropane Having Low Acidity

(75) Inventors: Vincent Luc Mutterer, Rosenau (FR); Jean-Philipe Tritz, Rixheim (FR)

Correspondence Address: ALBEMARLE CORPORATION, 451 FLORIDA STREET, BATON ROUGE, LA, 70801-1765, US

(73) Assignee: ALBEMARLE CORPORATION, Baton Rouge, LA (US)

(21) Appl. No.: 11/912,465

(22) PCT Filed: May 1, 2006
(86) PCT No.: PCT/US2006/016668

§ 371 Date: Oct. 24, 2007

Related U.S. Application Data
(60) Provisional application No. 60/677,277, filed on May 3, 2005.

(51) Int. Cl. C07C 21/14; C09K 3/00

(52) U.S. Cl. 570/200; 252/182.31

(57) ABSTRACT

This invention provides a process which comprises mixing at least one phenol with a 1-bromopropane product mixture to form a phenol-containing 1-bromopropane product mixture, and recovering a purified 1-bromopropane product from the phenol-containing 1-bromopropane product mixture. This invention also provides a process for preparing a 1-bromopropane product mixture from 1-propanol and hydrogen bromide, characterized by mixing at least one phenol with the 1-bromopropane product mixture to form a phenol-containing 1-bromopropane product mixture, and recovering a purified 1-bromopropane product from the phenol-containing 1-bromopropane product mixture.

### TECHNICAL FIELD

This invention relates to 1-bromopropane products having a reduced tendency for acid formation during storage, and to processes for producing such products.

### BACKGROUND

1-Bromopropane (also referred to as n-propyl bromide or propyl bromide) can be used as a degreasing agent, especially for degreasing metal parts, as well as in cleaning solutions for electrical circuit board production. In such applications, the presence of certain impurities in the 1-bromopropane is undesirable because the impurities have a detrimental effect on the substrates with which the 1-bromopropane is brought into contact. When manufactured, an initial 1-bromopropane product mixture contains impurities, including one or more of 2-bromopropane(isopropyl bromide), 1,2-dibromopropane, monobromoacetone, 1-propanol, propionaldehyde, propionic acid, water, and hydrogen bromide (HBr). Some of these impurities can, over time, degrade to yield acids, such as HBr and propionic acid. Thus, trace amounts of HBr are often present in finished 1-bromopropane products; further, the acidity increases over time in a finished 1-bromopropane product. Acidic species are detrimental to the performance qualities of 1-bromopropane as they can corrode or discolor the substrates to which the 1-bromopropane is applied. The art has tried to address this problem by adding acid scavenging compounds, such as 1,2-epoxides to the 1-bromopropane. Metal passivators, such as nitromethane and 1,3-dioxolane, have also been used. The problem with such additives is that there is a cost associated with their use, and they are consumed as they act to reduce acid content in the 1-bromopropane product. The consumption rate of the additive(s) can be such that, after a period of time, there is no further additive to reduce acid presence in the 1-bromopropane product. In this condition, the acid content of the 1-bromopropane can increase to deleterious levels. Thus, a better method for minimizing the acidic impurities in 1-bromopropane is needed, particularly one that does not compromise performance of the 1-bromopropane.

### SUMMARY OF THE INVENTION

This invention provides finished 1-bromopropane products having a reduced tendency to produce acidic species over time. In particular, the invention can provide finished 1-bromopropane products that meet the requirement of keeping the acidity below 10 ppm for at least 30 days at 60° C. without detracting from the performance of the finished 1-bromopropane product. Surprisingly, it has been found that contacting a 1-bromopropane product mixture with at least one phenol reduces or prevents the formation of acidity in a finished 1-bromopropane product produced therefrom. In particular, when a 1-bromopropane product mixture is treated with a phenol or phenols pursuant to this invention, the acidity of the finished 1-bromopropane product is reduced (in comparison to a finished 1-bromopropane product not so treated), and often the acidity of a phenol-treated finished 1-bromopropane product remains below 10 ppm for at least 30 days at 60° C. As used herein, the term “finished 1-bromopropane product” designates a 1-bromopropane product mixture that has been treated with at least one phenol, had the phenol(s) removed therefrom, and optionally has been subjected to further purification. Similarly, the term “1-bromopropane product mixture” as used herein denotes generally 1-bromopropane containing one or more of the impurities that are formed therewith during the course of manufacture of 1-bromopropane. More specifically, the 1-bromopropane product mixtures used in the processes of this invention generally contain 1-bromopropane and one or more of the impurities that are formed therewith during the course of manufacture of 1-bromopropane; the impurities are as described above.

This invention relates in part to processes for removing acidic and/or acid-forming species from 1-bromopropane product mixtures irrespective of the method of preparation of the 1-bromopropane product mixture. The processes of the invention result in the reduction of acidic species and/or acid-forming species, which may include propanol, propionaldehyde, and propionic acid. For example, 1-bromopropane product mixtures formed from 1-propanol and HBr typically contain propanol, propionaldehyde, and propionic acid, and 1-bromopropane product mixtures formed from propene and HBr often contain propionic acid. Thus, practicing the processes of this invention upon 1-bromopropane product mixtures made by such processes is advantageous.

Pursuant to this invention, a 1-bromopropane product mixture is mixed with at least one phenol. After the nixing, purification steps are taken to reduce the content of the phenol treatment products and unreacted phenol(s) in the 1-bromopropane product mixture. The finished 1-bromopropane product is obtained from the phenol-contacted 1-bromopropane product mixture. If desired, conventional additives such as acid scavengers and metal passivators can be added to the finished 1-bromopropane products of this invention.

An embodiment of this invention is a process which comprises mixing at least one phenol with a 1-bromopropane product mixture to form a phenol-containing 1-bromopropane product mixture, and recovering a purified 1-bromopropane product from the phenol-containing 1-bromopropane product mixture. The phenol(s) (I) are mixed with the 1-bromopropane product mixture in proportions of at least about 0.5 part by weight phenol per 1000 parts by weight 1-bromopropane product mixture, and (ii) are at least one mononuclear phenol having one or two hydroxyl groups directly bonded to the benzene ring and a total of 6 to 16 carbon atoms in the molecule, the at least one phenol being free of unsaturation other than the aromatic unsaturation of the benzene ring.

In this invention, as used herein, the term “phenol-containing 1-bromopropane product mixture” denotes a 1-bromopropane product mixture with which a phenol has been mixed, but which 1-bromopropane product mixture has not been separated from the phenol(s), i.e., a phenol or phenols, in one form or another, are present in the 1-bromopropane product mixture. Similarly, the term “purified 1-bromopropane product,” as used herein, denotes a 1-bromopropane product mixture that has been mixed with at least one phenol, and from which the phenol(s) have been separated. Usually and preferably, the purified 1-bromopropane product undergoes further purification to form a finished 1-bromopropane product; if the purified 1-bromopropane product is not further purified, the purified 1-bromopropane product is the finished 1-bromopropane product.

Another embodiment of this invention is a process for preparing a 1-bromopropane product mixture from 1-propanol and hydrogen bromide, characterized by mixing at least one phenol with the 1-bromopropane product mixture to form a phenol-containing 1-bromopropane product mixture, and recovering a purified 1-bromopropane product from the phenol-containing 1-bromopropane product mixture. The phenol(s) (I) are mixed with the 1-bromopropane product mixture in proportions of at least about 0.5 part by weight phenol per 1000 parts by weight 1-bromopropane product mixture, and (ii) are at least one mononuclear phenol having one or two hydroxyl groups directly bonded to the benzene ring and a total of 6 to 16 carbon atoms in the molecule, the at least one phenol being free of unsaturation other than the aromatic unsaturation of the benzene ring.

Still another embodiment of this invention is a composition comprising at least one phenol and a 1-bromopropane product mixture where the phenol(s) are present in proportions of at least about 0.5 part by weight phenol per 1000 parts by weight of 1-bromopropane product mixture. The phenol or phenols are at least one mononuclear phenol having one or two hydroxyl groups directly bonded to the benzene ring and a total of 6 to 16 carbon atoms in the molecule, the phenol(s) being free of unsaturation other than the aromatic unsaturation of the benzene ring.

Yet another embodiment of this invention is a composition which comprises a purified 1-bromopropane product which is a 1-bromopropane product mixture that has been contacted with at least one phenol in proportions of at least about 0.5 part by weight phenol per 1000 parts by weight of 1-bromopropane product mixture, and from which the phenol(s) have been removed. The phenol or phenols are at least one mononuclear phenol having one or two hydroxyl groups directly bonded to the benzene ring and a total of 6 to 16 carbon atoms in the molecule, the at least one phenol being free of unsaturation other than the aromatic unsaturation of the benzene ring.

These and other embodiments and features of this invention will be still further apparent from the ensuing description and appended claims.

### FURTHER DETAILED DESCRIPTION OF THE INVENTION

The term ppm means parts per million (wt/wt), unless specifically stated otherwise herein.

Preferred 1-bromopropane product mixtures used in this invention have a purity of at least about 90%, and more preferably a purity of at least about 95%, and still more preferably a purity of at least about 98%, the balance in each case being one or more impurities resulting from the process by which the 1-bromopropane product mixture was prepared.

A phenol or phenols can be mixed with a 1-bromopropane product mixture at almost any point after formation of the 1-bromopropane product mixture. While the phenol or phenols can be mixed with a crude 1-bromopropane product mixture (i.e., prior to any purification of the 1-bromopropane product mixture), normally and preferably the crude 1-bromopropane product mixture is washed with water and/or with an aqueous solution of at least one inorganic base prior to mixing with the phenol(s). More than one wash of the 1-bromopropane product mixture, or other purification steps, such as distillation, may be carried out before mixing the phenol(s) with the 1-bromopropane product mixture.

The phenol(s) are generally mixed with 1-bromopropane in solid form or liquid form. While the phenol or phenols can be mixed with a 1-bromopropane product mixture as a pre-formed solution, the use of a solution is not recommended because the solvent(s) used in forming the pre-formed solution can introduce undesirable impurities into the 1-bromopropane product mixture.

For the mixing of a phenol or phenols with a 1-bromopropane product mixture, the phenol(s) can be added to the 1-bromopropane product mixture, the 1-bromopropane product mixture can be added to the phenol(s), or the phenol(s) and the 1-bromopropane product mixture can be co-fed to a vessel or mixing zone. Preferably, the phenol or phenols are added to a 1-bromopropane product mixture.

Basic conditions are preferred for mixing a phenol or phenols with a 1-bromopropane product mixture. The pH is preferably in range of about 8 to about 10; higher pH values are acceptable but not necessary. By “basic conditions” it is meant that when water or an aqueous solution is stirred with a 1-bromopropane product mixture, and the aqueous and organic phases are allowed to separate, the pH of the aqueous phase is above 7. Basic conditions are typically achieved by performing one or more washes of a 1-bromopropane product mixture with an aqueous solution of an inorganic base, preferably an alkali metal base, more preferably an alkali metal hydroxide. An advantage to operation under basic conditions is that a water wash or a wash with an aqueous solution of an inorganic base will extract propionic acid from the purified 1-bromopropane product. Mixing of a phenol or phenols with a 1-bromopropane product mixture under acidic conditions is acceptable, but not preferred.

After the mixing, the phenol(s) are separated from the phenol-containing 1-bromopropane product mixture to recover a purified 1-bromopropane product from the phenol-containing 1-bromopropane product mixture. Conventional separation methods, such as solvent extraction, distillation, and washing, may be used; distillation is a preferred separation method for recovering a purified 1-bromopropane product from a phenol-containing 1-bromopropane product mixture. It is recommended and preferred that at least one wash of the purified 1-bromopropane product with water or with an aqueous solution of an inorganic base is carried out. Further purification steps such as another distillation or one or more additional washes with water and/or an aqueous solution of an inorganic base may be performed on the purified 1-bromopropane product. Although trace amounts of the phenol(s) (e.g., on the order of 0.5-10 ppm) sometimes remain in the purified 1-bromopropane product and the finished 1-bromopropane product, a finished 1-bromopropane product having such trace amounts of the phenol(s) usually have a non-volatile residue less than about 3 ppm when subjected to the non-volatile residue test described below.

In the processes of this invention, drying is normally the final step in the purification of a purified 1-bromopropane product, after all of the other purification steps, if any, have been performed. It is generally not desirable to mix a phenol or phenols with a 1-bromopropane product mixture after the drying step, because the phenol(s) will remain in the finished 1-bromopropane product.

Generally, drying of a 1-bromopropane product mixture pursuant to the processes of this invention is carried out by contacting the purified 1-bromopropane product with a drying agent. Suitable drying agents are well known in the art and include calcium chloride, magnesium sulfate, calcium sulfate, potassium carbonate, and the like. A preferred drying agent in the practice of this invention is calcium chloride. Preferably, drying continues until the water content of the purified 1-bromopropane product is less than about 100 ppm (wt/wt); more preferably, drying continues until the water content of the purified 1-bromopropane product is less than about 80 ppm (wt/wt). Often, the purified 1-bromopropane product after drying is a finished 1-bromopropane product.

In some preferred processes of the invention, a 1,2-epoxide is mixed with the purified 1-bromopropane product. The 1,2-epoxide can be mixed into a purified 1-bromopropane product before or after the drying step, or a portion of the 1,2-epoxide can be mixed with the purified 1-bromopropane product before the drying step, and another portion (the remainder) of the 1,2-epoxide can be mixed with the purified 1-bromopropane product after the drying step. The purified 1-bromopropane product, after drying and inclusion of a 1,2-epoxide, is usually a finished 1-bromopropane product.

In the practice of this invention, the phenol or phenols are at least one mononuclear phenol having one or two hydroxyl groups directly bonded to the benzene ring and a total of 6 to 16 carbon atoms in the molecule, the phenol being free of unsaturation other than the aromatic unsaturation of the benzene ring. Preferred mononuclear phenols are those which contain only carbon, hydrogen, and oxygen atoms in the molecule. A few non-limiting examples of such mononuclear phenols include phenol, catechol, resorcinol, hydroquinone, guaiacol, saligenin, carvacrol, thymol, o-cresol, m-cresol, p-cresol, o-ethylphenol, o-isopropylphenyl, 2,6-diisopropylphenol, o-tert-butylphenol, p-tert-butylphenol, and o-cyclohexylphenol.

In especially preferred embodiments of the invention, the mononuclear phenol is one or more substituted phenols of the formula

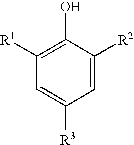

wherein:
A) R¹ and R² are both hydrogen atoms and R³ is an alkoxy group containing in the range of 1 to 5 (preferably in the range of 1 to 3) carbon atoms; or
B) R¹ is a hydroxyl group, R² is a hydrogen atom, and R³ is an alkyl group containing in the range of 1 to 5 carbon atoms (preferably tert-butyl or tert-amyl); or
C) R¹ and R² are both, independently, alkyl groups with the total number of carbon atoms in R¹ and R² being in the range of 5 to 10, with at least one of R¹ and R² being a tertiary alkyl group, and R³ is a hydrogen atom; or
D) R¹ and R² are both, independently, alkyl groups with the total number of carbon atoms in R¹ and R² being in the range of 5 to 10, with at least one of R¹ and R² being a tertiary alkyl group, and R³ is an alkyl group containing 1 or 2 carbon atoms.
   Non-limiting examples of the preferred substituted phenols of A), B), C), or D) above include 4-methoxyphenol, 4-ethoxyphenol, 4-propoxyphenol, 4-isopropoxyphenol, 4-butoxyphenol, 4-tert-butoxyphenol, 4-pentoxyphenol, 4-methyl-1,2-dihydroxybenzene, 4-ethyl-1,2-dihydroxybenzene, 4-propyl-1,2-dihydroxybenzene, 4-isobutyl-1,2-dihydroxybenzene, 4-tert-butyl-1,2-dihydroxybenzene, 4-tert-amyl-1,2-dihydroxybenzene, 2-methyl-6-tert-butylphenol, 2-ethyl-6-tert-butylphenol, 2-methyl-6-tert-amylphenol, 2-ethyl-6-tert-amylphenol, 2-isopropyl-6-tert-butylphenol, 2,6-di-tert-butylphenol, 2,6-di-tert-amylphenol, 2,4-dimethyl-6-tert-butylphenol, 2,4-diethyl-6-tert-butylphenol, 2-ethyl-6-tert-butylphenol, 2,4-dimethyl-6-tert-amylphenol, 2,4-diethyl-6-tert-amylphenol, 2-isopropyl-4-methyl-6-tert-butylphenol, 2,6-di-tert-butyl-4-methylphenol, 2,6-di-tert-butyl-4-ethylphenol, and 2,6-di-tert-amyl-4-methylphenol. Of these substituted phenols, 4-methoxyphenol, 4-tert-butyl-1,2-dihydroxybenzene, 2,6-di-tert-butylphenol, 2,6-di-tert-butyl-4-methylphenol, or a combination of any two or more of these is preferred. 2,6-Di-tert-butyl-4-methylphenol (also called 2,6-di-tert-butyl-p-cresol, sometimes abbreviated as BHT) is particularly preferred.

Mixtures of any two or more of the foregoing mononuclear phenols may be used in the practice of this invention.

Typically, the proportion of phenol(s) mixed with or contacted with a 1-bromopropane product mixture in the practice of this invention is at least about 0.5 part by weight phenol per 1000 parts by weight 1-bromopropane product mixture. Preferably, a phenol or phenols are mixed with a 1-bromopropane product mixture in proportions of about 0.5 to about 10 parts by weight phenol per 1000 parts by weight of 1-bromopropane product mixture; more preferably, the proportion of phenol mixed with a 1-bromopropane product mixture is in the range of about 1 to about 6 parts by weight of phenol per 1000 parts by weight of 1-bromopropane product mixture. Larger amounts of the phenol(s) can be used in the practice of this invention, but are not particularly desired, as excellent results are achieved with the amounts just described.

Various water-soluble inorganic bases may be used in the aqueous solution of inorganic base. Such inorganic bases include oxides, hydroxides, acetates, sulfates, carbonates, and bicarbonates of the alkali metals, alkaline earth metals, zinc, ammonium, and the like. Examples of suitable bases include sodium oxide, potassium oxide, magnesium oxide, calcium oxide, zinc oxide, ammonium hydroxide, lithium hydroxide, sodium hydroxide, potassium hydroxide, rubidium hydroxide, magnesium hydroxide, calcium hydroxide, barium hydroxide, zinc hydroxide, ammonium nitrate, ammonium sulfate, lithium acetate, lithium carbonate, sodium acetate, sodium bicarbonate, sodium carbonate, potassium acetate, potassium bicarbonate, potassium carbonate, potassium nitrite, potassium sulfite, rubidium carbonate, cesium acetate, cesium bicarbonate, magnesium carbonate, calcium carbonate, and zinc carbonate. Mixtures of two or more inorganic bases can be used. Alkali metal bases are preferred; more preferred are alkali metal hydroxides, and sodium hydroxide and potassium hydroxide are highly preferred as inorganic bases. Typically, the aqueous solution of inorganic base has in the range of about 15 to about 75 weight percent inorganic base, and the aqueous solution of inorganic base preferably has in the range of about 25 to about 55 weight percent inorganic base.

The term 1,2-epoxide does not mean that the ring must involve the carbon atoms in the 1- and 2-positions; instead this means that the epoxide (cyclic ether) has three atoms in the ring rather than 4 atoms in the ring. Examples of suitable 1,2-epoxides include alkylene oxides and/or cycloalkylene oxides of up to about 8 carbon atoms, including propylene oxide, butylene oxide, pentene oxide, hexene oxide, heptene oxide, octene oxide, cyclopentene oxide, cyclohexene oxide, methyl-1,2-cyclopentene oxide, and the like, or mixtures thereof. Preferably, the 1,2-epoxide is butylene oxide, irrespective of whether the butylene oxide is 1,2-epoxybutane or 2,3-epoxybutane or a mixture of both.

Typically, enough 1,2-epoxide is mixed with the purified 1-bromopropane product to make a concentration of 1,2-epoxide in the purified 1-bromopropane product in the range of about 10 to about 1000 ppm (wt/wt), and preferably in the range of about 100 to about 600 ppm (wt/wt). More preferably, the 1,2-epoxide concentration is in the range of about 250 to about 500 ppm (wt/wt); still more preferred is a 1,2-epoxide concentration in the purified 1-bromopropane product in the range of about 400 to about 500 ppm (wt/wt).

The use of a 1,2-epoxide is optional and unnecessary in the practice of this invention. After long periods of storage and/or exposure to high temperature (e.g., outdoor summer temperatures), the presence of a 1,2-epoxide in a finished 1-bromopropane product may prove useful, but absent such conditions, the phenol treatment alone is sufficient to keep the acidity of a finished 1-bromopropane product within desirable limits.

Preferred processes of the invention include those in which a 1,2-epoxide is mixed with a purified 1-bromopropane product in an amount to make a concentration of about 250 ppm to about 500 ppm, especially where the 1,2-epoxide is butylene oxide; preferably, the phenol is 4-methoxyphenol, 4-tert-butyl-1,2-dihydroxybenzene, 2,6-di-tert-butylphenol, 2,6-di-tert-butyl-4-methylphenol, or a combination of any two or more of these. More preferably, the processes further comprise drying the purified 1-bromopropane product until the water content of the purified 1-bromopropane product is less than about 100 ppm.

Other preferred processes of the invention comprise those in which the phenol or phenols are mixed with the 1-bromopropane product mixture in proportions of about 1 to about 6 parts by weight phenol per 1000 parts by weight of 1-bromopropane product mixture, and wherein a 1,2-epoxide is mixed with a purified 1-bromopropane product in an amount to make a concentration of about 250 ppm to about 500 ppm, where the 1,2-epoxide is butylene oxide; preferably, the phenol is 4-methoxyphenol, 4-tert-butyl-1,2-dihydroxybenzene, 2,6-di-tert-butylphenol, 2,6-di-tert-butyl-4-methylphenol, or a combination of any two or more of these. More preferably, these processes further comprise drying the purified 1-bromopropane product until the water content of the purified 1-bromopropane product is less than about 100 ppm.

Still other preferred processes of the invention comprise those in which the phenol or phenols are mixed with a 1-bromopropane product mixture in proportions of about 1 to about 6 parts by weight phenol per 1000 parts by weight of 1-bromopropane product mixture, wherein the phenol is 4-methoxyphenol, 4-tert-butyl-1,2-dihydroxybenzene, 2,6-di-tert-butylphenol, 2,6-di-tert-butyl-4-methylphenol, or a combination of any two or more of these, and wherein a 1,2-epoxide is mixed with the purified 1-bromopropane product in an amount to make a concentration of about 250 ppm to about 500 ppm. More preferably, the processes further comprise drying the purified 1-bromopropane product until the water content of the purified 1-bromopropane product is less than about 100 ppm.

In especially preferred processes of this invention, only water, one or more aqueous solutions of at least one inorganic base, and the phenol(s) are brought into contact with a 1-bromopropane product mixture. It is preferred that only water and one or more aqueous solutions of at least one inorganic base are brought into contact with the purified 1-bromopropane product.

During the mixing of a phenol or phenols with a 1-bromopropane product mixture, pursuant to this invention, a new composition of matter is formed, comprising at least one phenol and the 1-bromopropane product mixture, with the phenol(s) present in proportions of at least about 0.5 part by weight phenol per 1000 parts by weight of 1-bromopropane product mixture; this composition is a phenol-containing 1-bromopropane product mixture. It is to be understood that the composition encompasses the phenol(s) in whatever form into which the phenol(s) may be transformed when combined with a 1-bromopropane product mixture, and the 1-bromopropane product mixture.

Other preferred compositions of the invention comprising at least one phenol and a 1-bromopropane product mixture include those in which the phenol(s) are present in proportions of about 0.5 to about 10 parts by weight per 1000 parts by weight of 1-bromopropane product mixture. Still more preferred compositions of the invention comprising at least one phenol and a 1-bromopropane product mixture are those in which the phenol is present in proportions of about 1 to about 6 parts by weight per 1000 parts by weight of 1-bromopropane product mixture. Even more preferred compositions of the invention comprising at least one phenol and a 1-bromopropane product mixture are those in which the phenol or phenols are 4-methoxyphenol, 4-tert-butyl-1,2-dihydroxybenzene, 2,6-di-tert-butylphenol, 2,6-di-tert-butyl-4-methylphenol, or a combination of any two or more of these. In especially preferred compositions of the invention comprising at least one phenol and a 1-bromopropane product mixture, only the phenol(s) and the 1-bromopropane product mixture are present in the composition.

After the mixing of phenol(s) with a 1-bromopropane product mixture and the subsequent removal of the phenol(s) in the processes of this invention, a purified 1-bromopropane product that has been contacted with at least one phenol is produced. This purified 1-bromopropane product which is a 1-bromopropane product mixture that has been contacted with at least one phenol in proportions of at least about 0.5 part by weight phenol per 1000 parts by weight of 1-bromopropane product mixture, and from which the phenol(s) have been removed, is a new composition. The composition comprising a purified 1-bromopropane product can be formed in the processes of this invention after the mixing of a phenol or phenols with a 1-bromopropane product mixture and the subsequent separation of the phenol(s) from the phenol-containing 1-bromopropane product mixture. Preferably, the water content of the composition comprising a purified 1-bromopropane product is less than about 100 ppm; more preferably, the water content of the composition is less than about 80 ppm.

Preferred compositions of the invention comprising a purified 1-bromopropane product include those in which a 1,2-epoxide is present in the composition in a concentration of about 250 ppm to about 500 ppm, and wherein the 1,2-epoxide is butylene oxide. More preferably, the water content of the composition is less than about 100 ppm; still more preferably, the water content of the composition is less than about 80 ppm. In especially preferred compositions of the invention comprising a purified 1-bromopropane product, only the purified 1-bromopropane product and at least one 1,2-epoxide are present in the composition; if a 1,2-epoxide is not present in the composition, it is preferred that only the purified 1-bromopropane product is present in the composition.

In addition to the purified 1-bromopropane product, one or more other substances, although unnecessary, may be present in the composition that comprises a purified 1-bromopropane product. Such additives include 1,2-epoxides, nitroalkanes (e.g., nitromethane, nitroethane, etc.), N-alkylmorpholines, amines, dioxanes, dioxolanes, and other known stabilizers for 1-bromopropane. Surfactants, dyes, and other non-stabilizer components may optionally be included in the composition, provided no such component contributes substantially to an increase in the acidity of the composition, especially to an acidity above 10 ppm after 30 days at 60° C.

The following examples are presented for purposes of illustration, and are not intended to impose limitations on the scope of this invention.

The 60° C. stability test (also sometimes called the acidity test) used in the following Examples was conducted as follows: A quantity of about 160 grams of the finished 1-bromopropane product to be tested was placed in a 4 fluid ounce (118 mL) Boston Round screw cap bottle. The Teflon® (polymer-lined cap for the bottle was applied without excluding air from the free head space. The capped bottle was held in a 60° C. oven for 30 days, opening it once at 10 days to take a sample for analysis. The sample was then allowed to cool to room temperature before determining the acidity. The analysis for acidity involved shaking 80-120 grams of the cooled test sample with 30 to 50 mL of ultra pure water followed by phase separation and titration of the aqueous phase with 0.01 N NaOH to the phenolphthalein endpoint. This acidity analysis will show a positive result for HBr, as well as carboxylic acids such as propionic acid.

The gas chromatography analyses of the finished 1-bromopropane products in the following Examples were performed on a Hewlett-Packard 5890 gas chromatograph equipped with a split injector, flame ionization detector and a 30M×0.53 mm×3 μm DB-624 capillary column operating at 35° C. The temperature was held at 35 ° C. for 8 minutes, then raised at 10° C./min. to 230° C., final time3 minutes. The column head pressure was 4.5 psig (1.32×10⁵ Pa) and the total flow of He was 75 mL/minute. An injection volume of 0.5 μL of neat sample was used. The amounts of 2-bromopropane, propanol, propionaldehyde, and propionic acid reported in the Examples below were determined by gas chromatography (GC), and are based on GC area percent, unless otherwise stated.

A non-volatile residue test was performed on the finished 1-bromopropane products produced in some of the following Examples below. The procedure for this test was as follows: To a dry evaporation dish of known weight was added 100 mL of the sample to be tested. The dish was weighed again and placed under a heat lamp until the sample had evaporated to dryness. The dish was placed in a 105° C. oven for 1 hour, cooled in a dessicator, and weighed a final time. Non-volatile residue, in parts per million wt/wt, was calculated from the ratio of the final net weight to the starting net weight.

In the following Examples, the water content of the finished 1-bromopropane products was determined by coulometric Karl Fisher analysis.

Comparative Example A illustrates a typical process for preparing 1-bromopropane products from propanol and HBr, and Table A shows the amounts of impurities in a finished 1-bromopropane product at the end of a process and after storage when the 1-bromopropane product mixture is not mixed with a phenol. Table 1 (from Example 1) shows the beneficial effects of the inclusion of a phenol (Runs 1 and 2) on a finished 1-bromopropane product, in comparison to a 1-bromopropane product which has not been contacted with a phenol (Run 3, comparative). Examples 2 and 3 show processes of the invention.

### COMPARATIVE EXAMPLE A

A finished 1-bromopropane product was prepared from 1-propanol and HBr as follows:
(1) 1-Propanol (9000 L) was charged to a 16000 L reactor. A maximum HBr flow (200 m³·h⁻¹) to the reactor was started while the temperature of the cooled and agitated mixture in the reactor was kept at <45° C. After the reaction was complete, the mixture was cooled to 30° C. Water (300 L) was added, and the mixture was agitated. Agitation was stopped, and the mixture was allowed to settle. The resultant organic and aqueous phases were separated; the aqueous phase was discarded.
(2) While agitating the organic phase (the 1-bromopropane product mixture), aqueous NaOH (48%, 500 L) and water (100 L) were added to organic phase. Agitation was stopped, the aqueous and organic layers were allowed to separate, and the aqueous layer was discarded.
(3) Distillation of the organic layer (12500 kg) was started; the distillation was performed using 6 bar steam (165° C.) in the distillation jacket. The distilled organic layer was made to flow into agitated water (1000 kg), until the temperature in the distillation column head reached 80° C. Aqueous NaOH (23%) was added to the mixture of distillate (12000 kg) and water (1000 kg) until the pH was between 8 and 10. The mixture was agitated. Once agitation was stopped, the mixture was allowed to settle. The resultant organic and aqueous phases were separated; the aqueous phase was discarded.
(4) Water was added to the organic phase and the mixture was agitated. Aqueous NaOH (23 wt %) was again added to the mixture until the pH was between 8 and 10. The mixture was agitated. Once agitation was stopped, the mixture was allowed to settle. The resultant aqueous and organic phases were separated, and the aqueous phase was discarded.
(5) The organic phase was dried by passing it through a column containing CaCl₂ until the final water content was less than 80 ppm.

No phenol or butylene oxide was added during or after the preparation. Results for the 1-bromopropane product initially as well as after 10 days and after 30 days at 60° C. are summarized in Table A, as amounts present in the 1-bromopropane product.

TABLE A

| | Amount | | |
|---|---|---|---|
| | present | Amount | Amount |
| | at start of | present after | present after |
| | stability test | 10 days at 60° C. | 30 days at 60° C. |
| 1-Bromopropane | 99.92% | 99.87% | 99.85% |
| 2-Bromopropane | 173 ppm | 260 ppm | 332 ppm |
| Propanol | 175 ppm | 116 ppm | 910 ppm |
| Acidity | 5 ppm | 216 ppm | 150 ppm |
| Propionaldehyde | 10 ppm | 3 ppm | 2 ppm |
| Propionic acid | <1 ppm | 7 ppm | 8 ppm |

*Acidity is reported as ppm HBr.

### EXAMPLE 1

A preparation as described in Comparative Example A was carried out to form a finished 1-bromopropane product. No 2,6-di-tert-butyl-4-methylphenol (BHT) was added during the preparation. Three runs to test the effectiveness of stabilizers in the 1-bromopropane product were carried out. In Runs 1 and 2, an amount of BHT was added to a known amount of the 1-bromopropane product; in Run 3, butylene oxide (abbreviated BOX in Table 1 below) was added to a known amount of 1-bromopropane product. The stability test was performed on all three samples. No 1,2-epoxide was present in Run 1 or in Run 2; Run 3, which did not have any phenol added, is a comparative example. The amount of BHT or butylene oxide (BOX) added to the 1-bromopropane product in each run is listed in Table 1. Results for the 1-bromopropane products initially as well as after 10 days and after 30 days at 60° C. are summarized in Table 1, as amounts present in the 1-bromopropane product.

TABLE 1

| | Amount | Amount | Amount |
|---|---|---|---|
| | present | present | present |
| | at start of | after 10 days at | after 30 days at |
| | stability test | 60° C. | 60° C. |
| Run 1 (1.242 g BHT per | | | |
| kg 1-bromopropane) | | | |
| 1-Bromopropane | 99.60% | 99.60% | 99.63% |
| 2-Bromopropane | 163 ppm | 162 ppm | 166 ppm |
| Propanol | 177 ppm | 176 ppm | 182 ppm |
| Acidity* | 3 ppm | 6.2 ppm | 1.8 ppm |
| Propionaldehyde | 11 ppm | 11 ppm | 11 ppm |
| Propionic acid | <1 ppm | <1 ppm | <1 ppm |
| BHT | 1242 ppm | 1242 ppm | 1242 ppm |
| Run 2 (2.436 g BHT per | | | |
| kg 1-bromopropane) | | | |
| 1-Bromopropane | 99.27% | 99.28% | 99.33% |
| 2-Bromopropane | 161 ppm | 160 ppm | 160 ppm |
| Propanol | 175 ppm | 175 ppm | 183 ppm |
| Acidity* | 4 ppm | 4.2 ppm | 2.4 ppm |
| Propionaldehyde | 11 ppm | 11 ppm | 11 ppm |
| Propionic acid | <1 ppm | <1 ppm | <1 ppm |
| BHT | 2436 ppm | 2436 ppm | 2436 ppm |
| Run 3 (0.5244 g BOX | | | |
| per kg 1-bromopropane)^{†} | | | |
| 1-Bromopropane | 99.88% | 99.87% | 99.87% |
| 2-Bromopropane | 161 ppm | 157 ppm | 159 ppm |
| Propanol | 175 ppm | 168 ppm | 163 ppm |
| Acidity* | 5 ppm | 40 ppm | 60 ppm |
| Propionaldehyde | 11 ppm | 7 ppm | 10 ppm |
| Propionic acid | <1 ppm | 11 ppm | 25 ppm |

*Acidity is reported as ppm HBr.
^{†}Comparative example.

As can be seen from the results in Table 1, in 1-bromopropane products that have been treated with BHT, propionic acid does not increase appreciably, nor does the acidity increase significantly, during the stability test in a finished 1-bromopropane product obtained from a phenol-treated 1-bromopropane product mixture, in contrast to either a 1-bromopropane product with butylene oxide (Table 1) or a 1-bromopropane product without a stabilizer (see Table A in the Comparative Example above).

### EXAMPLE 2

The preparation described above in Comparative Example A was carried out to form a finished 1-bromopropane product; in addition, 2,6-di-tert-butyl-4-methylphenol (BHT, 50 kg) was added to the 1-bromopropane product mixture prior to starting the distillation of the organic layer in Step (3); at the end of the distillation, some BHT (˜1000 ppm) was entrained in the purified 1-bromopropane product, as seen by GC. At the end of Step (5), enough butylene oxide was added to the purified 1-bromopropane product to make a butylene oxide concentration of about 450-500 ppm. BHT was detected in the finished 1-bromopropane product by GC, and its area percent indicated 2 ppm BHT was present. The finished 1-bromopropane product was subjected to the acidity test as described above. Results for the finished 1-bromopropane product initially as well as after 10, 12, 25, and 31 days at 60° C. are summarized in Table 2, as amounts present in the finished 1-bromopropane product. The finished 1-brompropane product was also subjected to the non-volatile residue test as described above, and the non-volatile residue was found to be 3 ppm.

TABLE 2

| | | Amount present | Amount present | Amount present |
|---|---|---|---|---|
| | Amount present at | after 12 days at | after 25 days at | after 31 days at |
| | start of stability test | 60° C. | 60° C. | 60° C. |
| 1-Bromopropane | 99.85% | 99.86% | 99.80% | 99.80% |
| 2-Bromopropane | 180 ppm | 180 ppm | 180 ppm | 180 ppm |
| Propanol | 100 ppm | 90 ppm | 90 ppm | 90 ppm |
| Acidity* | 2.9 ppm | 4.1 ppm | 2.8 ppm | 2.4 ppm |
| Propionaldehyde | 11 ppm | 10 ppm | 10 ppm | 10 ppm |
| Propionic acid | <1 ppm | <1 ppm | <1 ppm | <1 ppm |
| BHT | 2 ppm | 2 ppm | 2 ppm | 2 ppm |

*Acidity is reported as ppm HBr.

It can be seen from Table 2 that propionaldehyde and propionic acid do not increase appreciably, nor does the acidity increase significantly during the stability test in a finished 1-bromopropane product obtained from a phenol-treated 1-bromopropane product mixture.

### EXAMPLE 3

A preparation as described in Example 2 was carried out, inclusive of the addition of butylene oxide, to form a finished 1-bromopropane product. The amount of BHT added was 8 g per 1489 g of 1-bromopropane product mixture; no BHT was detected in the 1-bromopropane after the distillation step, nor were any new compounds detected in the 1-bromopropane product mixture by GC. Results for the finished 1-bromopropane product initially as well as after 10 days and after 30 days at 60° C. are summarized in Table 3, as amounts present in the finished 1-bromopropane product. The finished 1-bromopropane product was also subjected to the non-volatile residue test as described above, and the non-volatile residue was 3 ppm.

TABLE 3

| | Amount | | | | | |
|---|---|---|---|---|---|---|
| | present | | Amount | | Amount | |
| | at start of | | present after | | present after | |
| | stability test | | 10 days at 60° C. | | 30 days at 60° C. | |
| 1-Bromopropane | 99.93% | | 99.93% | | 99.92% | |
| 2-Bromopropane | 152 | ppm | 152 | ppm | 161 | ppm |
| Propanol | 4 | ppm | 4 | ppm | <1 | ppm |
| Acidity* | 3 | ppm | 4 | ppm | 3.3 | ppm |
| Propionaldehyde | 1 | ppm | 2 | ppm | 1 | ppm |
| Propionic acid | 0 | | 0 | | 0 | |
| BHT | <1 | ppm | <1 | ppm | <1 | ppm |

*Acidity is reported as ppm HBr.

It is to be understood that the reactants and components referred to by chemical name or formula anywhere in this document, whether referred to in the singular or plural, are identified as they exist prior to coming into contact with another substance referred to by chemical name or chemical type (e.g., another reactant, a solvent, or etc.). It matters not what preliminary chemical changes, transformations and/or reactions, if any, take place in the resulting mixture or solution or reaction medium as such changes, transformations and/or reactions are the natural result of bringing the specified reactants and/or components together under the conditions called for pursuant to this disclosure. Thus the reactants and components are identified as ingredients to be brought together in connection with performing a desired chemical operation or reaction or in forming a mixture to be used in conducting a desired operation or reaction. Also, even though an embodiment may refer to substances, components and/or ingredients in the present tense (“is comprised of”, “comprises”, “is”, etc.), the reference is to the substance, component or ingredient as it existed at the time just before it was first contacted, blended or mixed with one or more other substances, components and/or ingredients in accordance with the present disclosure.

Also, even though the claims may refer to substances in the present tense (e.g., “comprises”, “is”, etc.), the reference is to the substance as it exists at the time just before it is first contacted, blended or mixed with one or more other substances in accordance with the present disclosure.

Except as may be expressly otherwise indicated, the article “a” or “an” if and as used herein is not intended to limit, and should not be construed as limiting, the description or a claim to a single element to which the article refers. Rather, the article “a” or “an” if and as used herein is intended to cover one or more such elements, unless the text expressly indicates otherwise.

Each and every patent or other publication or published document referred to in any portion of this specification is incorporated in toto into this disclosure by reference, as if fully set forth herein.

This invention is susceptible to considerable variation in its practice.

1. A process which comprises mixing at least one phenol with a 1-bromopropane product mixture to form a phenol-containing 1-bromopropane product mixture, and recovering a purified 1-bromopropane product from said phenol-containing 1-bromopropane product mixture, wherein (I) said phenol is mixed with said 1-bromopropane product mixture in proportions of at least about 0.5 part by weight phenol per 1000 parts by weight 1-bromopropane product mixture, and (ii) said phenol is at least one mononuclear phenol having one or two hydroxyl groups directly bonded to the benzene ring and a total of 6 to 16 carbon atoms in the molecule, said at least one phenol being free of unsaturation other than the aromatic unsaturation of the benzene ring.

2. A process for preparing a 1-bromopropane product mixture from 1-propanol and hydrogen bromide, characterized by mixing at least one phenol with said 1-bromopropane product mixture to form a phenol-containing 1-bromopropane product mixture, and recovering a purified 1-bromopropane product from said phenol-containing 1-bromopropane product mixture, wherein (I) said phenol is mixed with said 1-bromopropane product mixture in proportions of at least about 0.5 part by weight phenol per 1000 parts by weight 1-bromopropane product mixture, and (ii) said phenol is at least one mononuclear phenol having one or two hydroxyl groups directly bonded to the benzene ring and a total of 6 to 16 carbon atoms in the molecule, said at least one phenol being free of unsaturation other than the aromatic unsaturation of the benzene ring.

3-4. (canceled)

5. A process as in claim 1 wherein said phenol contains only carbon, hydrogen, and oxygen atoms in the molecule.

6. A process as in claim 1 wherein said phenol is one or more substituted phenols of the formula 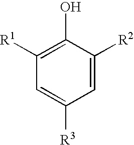
wherein:
A) R¹ and R² are both hydrogen atoms and R³ is an alkoxy group containing in the range of 1 to 5 (preferably in the range of 1 to 3) carbon atoms; or
B) R¹ is a hydroxyl group, R² is a hydrogen atom, and R³ is an alkyl group containing in the range of 1 to 5 carbon atoms (preferably tert-butyl or tert-amyl); or
C) R¹ and R² are both, independently, alkyl groups with the total number of carbon atoms in R¹ and R² being in the range of 5 to 10, with at least one of R¹ and R² being a tertiary alkyl group, and R³ is a hydrogen atom; or
D) R¹ and R² are both, independently, alkyl groups with the total number of carbon atoms in R¹ and R² being in the range of 5 to 10, with at least one of R¹ and R² being a tertiary alkyl group, and R³ is an alkyl group containing 1 or 2 carbon atoms.

7. A process as in claim 1 wherein said phenol is selected from the group consisting of 4-methoxyphenol, 4-tert-butyl-1,2-dihydroxybenzene, 2,6-di-tert-butylphenol, 2,6-di-tert-butyl-4-methylphenol, or a combination of any two or more of the foregoing.

8-16. (canceled)

17. A process as in claim 1 wherein only water, one or more aqueous solutions of at least one inorganic base, and said phenol are brought into contact with the 1-bromopropane product mixture.

18-23. (canceled)

24. A process as in claim 1 wherein said process further comprises drying the purified 1-bromopropane product until it has a water content of less than about 100 ppm (wt/wt).

25. (canceled)

26. A process as in claim 1 wherein at least one 1,2-epoxide is mixed with the purified 1-bromopropane product.

27. (canceled)

28. A process as in claim 26 wherein said 1,2-epoxide is butylene oxide.

29-35. (canceled)

36. A process as in claim 1 further comprising forming a finished 1-bromopropane product from said purified 1-bromopropane product, wherein the finished 1-bromopropane product has an acidity of less than about 10 ppm as ppm HBr after 30 days at 60° C.

37. (canceled)

38. A process as in claim 1 wherein the 1-bromopropane product mixture has a purity of at least about 90%.

39-42. (canceled)

43. A composition comprising at least one phenol and a 1-bromopropane product mixture wherein (I) said phenol is present in proportions of at least about 0.5 part by weight phenol per 1000 parts by weight of 1-bromopropane product mixture, and (ii) said phenol is at least one mononuclear phenol having one or two hydroxyl groups directly bonded to the benzene ring and a total of 6 to 16 carbon atoms in the molecule, said at least one phenol being free of unsaturation other than the aromatic unsaturation of the benzene ring.

44. A composition as in claim 43 wherein said phenol contains only carbon, hydrogen, and oxygen atoms in the molecule.

45. A composition as in claim 43 wherein said phenol is one or more substituted phenols of the formula 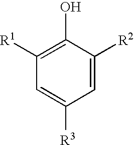
wherein:
A) R¹ and R² are both hydrogen atoms and R³ is an alkoxy group containing in the range of 1 to 5 (preferably in the range of 1 to 3) carbon atoms; or
B) R¹ is a hydroxyl group, R² is a hydrogen atom, and R³ is an alkyl group containing in the range of 1 to 5 carbon atoms (preferably tert-butyl or tert-amyl); or
C) R¹ and R² are both, independently, alkyl groups with the total number of carbon atoms in R¹ and R² being in the range of 5 to 10, with at least one of R¹ and R² being a tertiary alkyl group, and R³ is a hydrogen atom; or
D) R¹ and R² are both, independently, alkyl groups with the total number of carbon atoms in R¹ and R² being in the range of 5 to 10, with at least one of R¹ and R² being a tertiary alkyl group, and R³ is an alkyl group containing 1 or 2 carbon atoms.

46. A composition as in claim 43 wherein said phenol is selected from the group consisting of 4-methoxyphenol, 4-tert-butyl-1,2-dihydroxybenzene, 2,6-di-tert-butylphenol, 2,6-di-tert-butyl-4-methylphenol, or a combination of any two or more of the foregoing.

47-49. (canceled)

50. A composition as in claim 43 wherein the 1-bromopropane product mixture has a purity of at least about 90%.

51. (canceled)

52. A composition as in claim 43 wherein only said phenol and the 1-bromopropane product mixture are present in the composition.

53. A composition which comprises a purified 1-bromopropane product which is a 1-bromopropane product mixture that has been contacted with at least one phenol in proportions of at least about 0.5 part by weight phenol per 1000 parts by weight of 1-bromopropane product mixture, and from which said phenol has been removed, wherein said phenol is at least one mononuclear phenol having one or two hydroxyl groups directly bonded to the benzene ring and a total of 6 to 16 carbon atoms in the molecule, said at least one phenol being free of unsaturation other than the aromatic unsaturation of the benzene ring.

54. A composition as in claim 53 wherein a 1,2-epoxide is present, and wherein said 1,2-epoxide has a concentration in the composition of about 250 ppm to about 500 ppm.

55. A composition as in claim 54 wherein the 1,2-epoxide is butylene oxide.

56. A composition as in claim 53 wherein the composition has a water content of less than about 100 ppm.

57. (canceled)

58. A composition as in claim 53 wherein only the purified 1-bromopropane product and at least one 1,2-epoxide are present in said composition.

59. A composition as in claim 53 wherein only the purified 1-bromopropane product is present in said composition.

60. A composition as in claim 53 wherein said composition has an acidity of less than about 10 ppm as ppm HBr after 30 days at 60° C.

61. (canceled)
